(19) United States
(12) Patent Application Publication
(10) Pub. No.: US 2026/0224389 A1
(43) Pub. Date: Aug. 6, 2026

(54) FLUID COLLECTION DEVICES HAVING A DISTAL RECEPTACLE DISPOSED IN A DISTAL END REGION OF A FLUID IMPERMEABLE BARRIER, AND RELATED SYSTEM AND METHODS

(71) Applicant: PUREWICK CORPORATION, Covington, GA (US)

(72) Inventor: Aravind Arivazhagan, Tamilnadu (IN)

(21) Appl. No.: 19/046,047

(22) Filed: Feb. 5, 2025

(51) Int. Cl. A61F 5/455; A61F 5/44; A61M 1/00

(52) U.S. Cl. CPC A61F 5/4553; A61F 5/4404; A61M 1/71; A61M 2202/0014; A61M 2202/0496

(57) ABSTRACT

Embodiments disclosed herein are fluid collection devices including a fluid impermeable barrier that is generally flexible, at least one porous material, and a distal receptacle that is more rigid than the fluid impermeable barrier. The fluid impermeable barrier includes a proximal end region and a distal end region, and at least partially defines a chamber therein, an aperture in the proximal end region, and an opening extending therethrough between the distal end region and the proximal end region. The at least one porous material is disposed in the chamber, at least a portion of the at least one porous material positioned to extend across at least partially across the opening. The distal receptacle is positioned within the chamber in the distal end region of the fluid impermeable barrier and defines a reservoir in fluid communication with the opening through the at least one porous material.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters may be uncomfortable, painful, and may cause urinary tract infections.

Thus, users and manufacturers of fluid collection devices continue to seek new and improved devices, systems, and methods to collect urine.

### SUMMARY

Embodiments disclosed herein are fluid collection devices having a distal receptacle disposed in a distal end region of a fluid impermeable barrier, and related systems and methods. In an embodiment, a fluid collection device includes a fluid impermeable barrier that is generally flexible and has a proximal end region and a distal end region. The fluid impermeable barrier at least partially defines a chamber therein, an aperture in the proximal end region, and an opening extending therethrough between the distal end region and the proximal end region and configured to be positioned adjacent to a female urethra. The fluid collection device also includes at least one porous material disposed in the chamber, at least a portion of the at least one porous material positioned to extend across at least partially across the opening. The fluid collection device also includes a distal receptacle that is more rigid than the fluid impermeable barrier and positioned within the chamber in the distal end region of the fluid impermeable barrier or has the distal end region of the fluid impermeable barrier positioned within the distal receptacle. The distal receptacle defines a reservoir in fluid communication with the opening through the at least one porous material.

In an embodiment, a fluid collection system includes a fluid storage container configured to hold a fluid, the fluid collection device described above, and a vacuum source fluidly coupled to one or more of the fluid storage container or the fluid collection device via the conduit. The vacuum source is configured to draw fluid from the fluid collection device via the conduit.

In an embodiment, a method of assembling a fluid collection device includes providing a fluid impermeable barrier that is generally flexible and has a proximal end region and a distal end region. The fluid impermeable barrier at least partially defines a chamber therein, an aperture in the proximal end region, and an opening extending therethrough between the distal end region and the proximal end region and configured to be positioned adjacent to a female urethra. The method includes disposing a distal receptacle within the chamber in the distal end region of the fluid impermeable barrier, the distal receptacle being more rigid than the fluid impermeable barrier defining a reservoir in fluid communication with the opening. The method includes disposing at least one porous material within the chamber such that at least a portion of the at least one porous material extends at least partially across the opening and the reservoir of the distal receptacle is in fluid communication with the opening through the at least one porous material.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection device, according to an embodiment.

FIG. 1B is a side view of the fluid collection device of FIG. 1A.

FIG. 2A is an isometric view of a fluid impermeable barrier of the fluid collection device of FIG. 1A.

FIG. 2B is an isometric view of a porous material, a distal receptacle, and a conduit of the fluid collection device of FIG. 1A.

FIG. 2C is an isometric view of the conduit and the distal receptacle of the fluid collection device of FIG. 1A.

FIGS. 3A-3E are views of the distal receptacle of the fluid collection device of FIG. 1A.

FIG. 4A is a cross-sectional view of the fluid collection device of FIG. 1A along A-A of FIG. 1A.

FIG. 4B is a cross-sectional view of the distal end region of the fluid collection device of FIG. 1A along B-B of FIG. 1B.

FIG. 5 is a flow diagram of a method to manufacturing a fluid collection device, according to an embodiment.

FIG. 6 is a block diagram of a system for fluid collection, according to an embodiment.

### DETAILED DESCRIPTION

Embodiments disclosed herein are fluid collection devices having a generally flexible fluid impermeable barrier and a tapered distal receptacle that is more rigid that the fluid impermeable barrier and disposed in a distal end region of the fluid impermeable barrier. Embodiments of the fluid collection devices disclosed herein are configured to be positioned external to a user and collect fluids from an individual (e.g., an external catheter). The fluids collected by the fluid collection devices may include urine. The fluids collected by the fluid collection devices may also include at least one of vaginal discharge, penile discharge, reproductive fluids, blood, sweat, or other bodily fluids. The fluid collection devices disclosed herein include a fluid impermeable barrier that at least partially defines a chamber. The fluid impermeable barrier also defines an opening extending therethrough that is configured to be positioned adjacent to a female urethra. The fluid collection devices disclosed herein may include a porous material disposed in the chamber. The fluid collection devices disclosed herein include a distal receptacle positioned in the distal end region of the chamber of the fluid impermeable barrier. The distal receptacle is more rigid that the flexible fluid impermeable barrier (e.g., the distal receptacle may be substantially rigid).

In conventional external catheters, the urine collection area may be reduced when body parts (such as the gluteal cleft) press against the flexible material of the external catheter. In fluid collection devices disclosed herein, a distal receptacle that is more rigid than the fluid impermeable barrier is disposed in the distal end region of the fluid impermeable barrier. The distal receptacle includes a reservoir. The positioning and material of the distal receptacle creates more space for urine collection at the distal end region (e.g. base) of the fluid collection device within the distal receptacle. For example, while the fluid impermeable barrier may be formed of a generally flexible material (e.g., elastic rubber or silicone), the distal receptacle may be formed of a more rigid material (e.g. polypropylene or other semi-rigid or substantially rigid plastic). The distal receptacle ensures that the reservoir of the fluid collection device is not affected by external pressure from the body of the user. Accordingly, The distal receptacle provides a stable collection reservoir for urine collection in the fluid collection device. Fluid collection devices disclosed herein improve the fluid collection area capacity and also the user experience, according to one or more embodiments. For example, the distal receptacle includes a generally curved wedge shape that is comfortable for the user and may be easily inserted and remain in place for extended time periods compared to conventional external catheters. The fluid collection devices also include a conduit having a channel extending between an inlet and outlet thereof. The inlet is configured to be secured to the distal receptacle at least proximate to the reservoir, according to one or more embodiments. The conduit is configured to be fluidly coupled to a fluid storage (vessel or container).

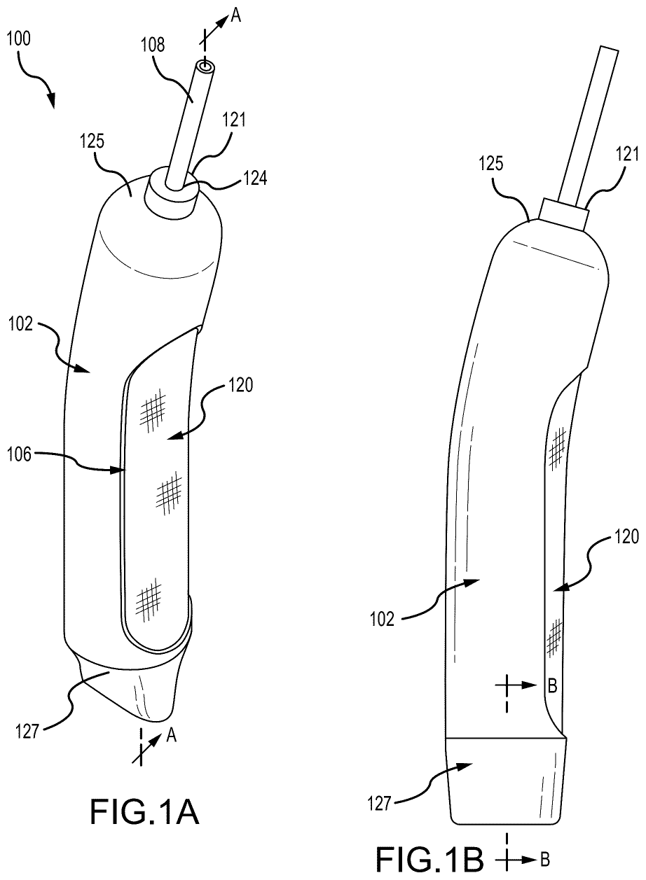

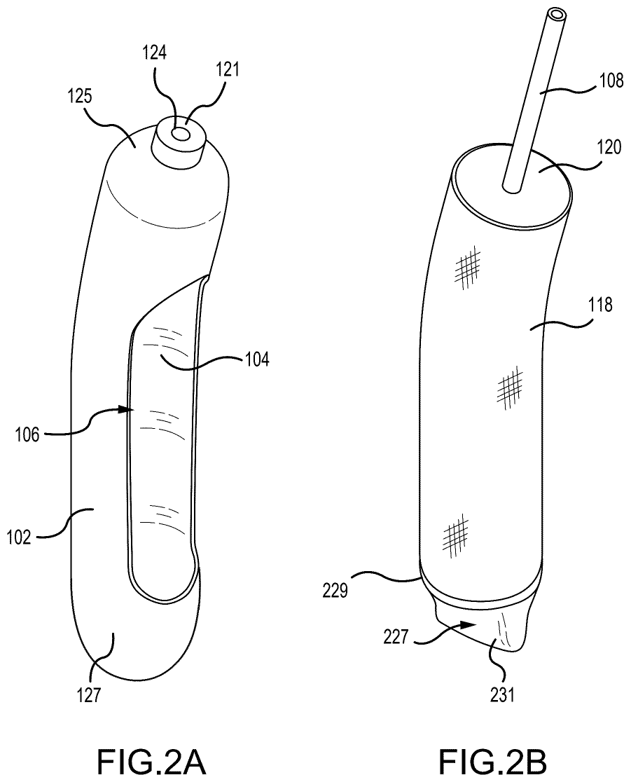

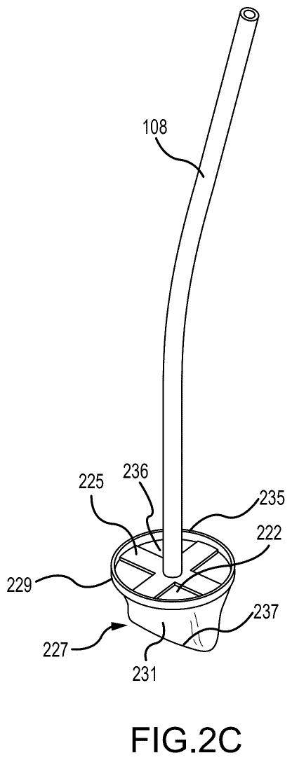

FIG. 1A is an isometric view and FIG. 1B is a side view of a fluid collection device 100, according to an embodiment. The fluid collection device 100 is an example of a female fluid collection device (e.g., external catheter) for receiving and collecting fluid(s) from a female. The fluid collection device 100 includes a fluid impermeable barrier 102, porous material 120 (e.g., wicking material) disposed in a chamber within the fluid impermeable barrier 102, a distal receptacle 227 (not visible in FIGS. 1A-1B, shown in FIG. 2B-4E) and an optional conduit 108 at least partially disposed within the chamber.

The fluid impermeable barrier 102 has a proximal end region 125 and a distal end region 127. The fluid impermeable barrier 102 may an aperture 124 in the proximal end region 125 and an opening 106 extending therethrough between the distal end region 127 and the proximal end region 125. The opening 106 is configured to be positioned adjacent to a female urethra. In some embodiments, the fluid impermeable barrier 102 include a proximal neck 121 extending proximally from the proximal end region 125 of the fluid impermeable barrier 102. The conduit 108 extends through the aperture 124 in the neck 121 and may be adhesively bonded to the neck 121, according to an embodiment.

FIG. 2A is an isometric view of a fluid impermeable barrier 102 of the fluid collection device 100 with the conduit 108, the porous material 120, and the distal receptacle 227 removed. The fluid impermeable barrier 102 at least partially defines a chamber 104 (e.g., interior region) and the opening 106. For example, the interior surface(s) of the fluid impermeable barrier 102 may at least partially define the chamber 104 within the fluid collection device 100. The fluid impermeable barrier 102 is configured to, with the distal receptacle 227, temporarily stores the fluid(s) in the chamber 104. The fluid impermeable barrier 102 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 102 substantially prevents the fluid(s) from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an outer surface 105 of the fluid impermeable barrier 102 may be formed from a soft and/or smooth material, thereby reducing chaffing.

In some examples, the fluid impermeable barrier 102 may be tubular (ignoring the opening), such as substantially cylindrical (as shown), oblong, prismatic, or flattened tubes. During use, the outer surface of the fluid impermeable barrier 102 may contact the wearer. The fluid impermeable barrier 102 may be sized and shaped to fit in the gluteal cleft between the legs of a female wearer.

The opening 106 provides an ingress route for fluids to enter the chamber 104. The opening 106 may be defined by the fluid impermeable barrier 102 such as by an inner edge of the fluid impermeable barrier 102. For example, the opening 106 is formed in and extends through the fluid impermeable barrier 102, from the outer surface 105 to the inner surface 103, thereby enabling fluid(s) to enter the chamber 104 from outside of the fluid collection device 100. The opening 106 may be an elongated hole in the fluid impermeable barrier 102. For example, the opening 106 may be defined as a cut-out in the fluid impermeable barrier 102. The opening 106 may be located and shaped to be positioned adjacent to a female urethra.

The fluid collection device 100 may be positioned proximate to the female urethra and urine may enter the chamber of the fluid collection device 100 via the opening 106. The fluid collection device 100 is configured to receive the fluid(s) into the chamber 104 via the opening 106. When in use, the opening 106 may have an elongated shape that extends from a first location below the urethral opening (e.g., at or near the anus or the vaginal opening) to a second location above the urethral opening (e.g., at or near the top of the vaginal opening or the pubic hair).

The opening 106 may have an elongated shape because the space between the legs of a female is relatively small when the legs of the female are closed, thereby only permitting the flow of the fluid(s) along a path that corresponds to the elongated shape of the opening 106 (e.g., longitudinally extending opening). The opening 106 in the fluid impermeable barrier 102 may exhibit a length that is measured along the longitudinal axis of the fluid collection device 100 that may be at least about 10% of the length of the fluid collection device 100, such as about 25% to about 50%, about 40% to about 60%, about 50% to about 75%, about 65% to about 85%, or about 75% to about 95% of the length of the fluid collection device 100.

The opening 106 in the fluid impermeable barrier 102 may exhibit a width that is measured transverse to the longitudinal axis of the fluid collection device 100 that may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid through the porous material 120 and into the conduit 108. In some examples, the opening 106 may be vertically oriented (e.g., having a major axis parallel to the longitudinal axis of the device 100). In some examples (not shown), the opening 106 may be horizontally oriented (e.g., having a major axis perpendicular to the longitudinal axis of the device 100). In an example, the fluid impermeable barrier 102 may be configured to be attached to the wearer, such as adhesively attached (e.g., with a hydrogel adhesive) to the wearer. According to an example, a suitable adhesive is a hydrogel layer.

In some examples, the fluid impermeable barrier 102 may define the aperture 124 sized to receive the conduit 108. The conduit 108 may be disposed in the chamber 104 via the aperture 124. The aperture 124 may be sized and shaped to form an at least substantially fluid tight seal against the conduit 108 or the at least one tube thereby substantially preventing the fluid(s) from escaping the chamber 104. As provided above, the fluid impermeable barrier 102 may include a proximal neck 121 extending proximally from the proximal end region 125 of the fluid impermeable barrier 102. The neck 121 may be substantially solid with only the aperture 124 sized to receive the conduit 108 extending therethrough. In some embodiments, the aperture 124 is sized smaller than the diameter of the conduit 108 such that the neck 121 friction fits around the conduit 108. In some embodiments, the conduit 108 is adhesively bonded to the neck 121.

The fluid impermeable barrier 102 may include markings (not shown) thereon, such as one or more markings to aid a wearer in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

FIG. 2B is an isometric view of the porous material 120, the distal receptacle 227, and the conduit 108 of the fluid collection device 100 secured together and removed from the fluid impermeable barrier 102. In some embodiments, the porous material 120, the distal receptacle 227, and the conduit 108 may be assembled and secured together before the assembly is inserted into and secured to the fluid impermeable barrier 102.

The fluid collection device 100 includes the porous material 120 disposed in the chamber 104. The porous material 120 may cover at least a portion (e.g., all) of the opening 106. The porous material 120 is exposed to the environment outside of the chamber 104 through the opening 106. The porous material 120 may be configured to move any fluid away from the opening 106, thereby preventing the fluid from escaping the chamber 104. The permeable properties referred to herein may be wicking, capillary action, diffusion, or other similar properties or processes, and are referred to herein as “permeable” and/or “wicking.” Such “wicking” may not include absorption of fluid into the porous material. Put another way, substantially no absorption of fluid into the material may take place after the material is exposed to the fluid and removed from the fluid for a time. While no absorption is desired, the term “substantially no absorption” may allow for nominal amounts of absorption of fluid into the porous material (e.g., absorbency), such as less than about 10 wt % of the dry weight of the porous material, less than about 7 wt %, less than about 5 wt %, less than about 3 wt %, less than about 2 wt %, less than about 1 wt %, or less than about 0.5 wt % of the dry weight of the porous material. The porous material 120 may also wick the fluid generally towards an interior of the chamber 104, as discussed in more detail below. The porous material 120 may include one or more (e.g., both) of a fluid permeable membrane 118 and/or a fluid permeable body 120. However, in some embodiments, it is noted that the porous material 120 may include an absorption material (e.g., hydrophilic material) instead of a wicking material.

The fluid collection device 100 may include the fluid permeable membrane 118 disposed in the chamber 104. The fluid permeable membrane 118 may cover at least a portion (e.g., all) of the opening 106. The fluid permeable membrane 118 may be composed to wick fluid away from the opening 106, thereby preventing the fluid from escaping the chamber 104.

The fluid permeable membrane 118 may include any material that may wick the fluid. For example, the fluid permeable membrane 118 may include fabric, such as a gauze (e.g., a silk, linen, or cotton gauze), another soft fabric, or another smooth fabric. Forming the fluid permeable membrane 118 from gauze, soft fabric, and/or smooth fabric may reduce chaffing caused by the fluid collection device 100.

The fluid collection device 100 may include the fluid permeable body 120 disposed in the chamber 104. The fluid permeable body 120 is configured to support the fluid permeable membrane 118 since the fluid permeable membrane 118 may be formed from a relatively foldable, flimsy, or otherwise easily deformable material. For example, the fluid permeable body 120 may be positioned such that the fluid permeable membrane 118 is disposed between the fluid permeable body 120 and the fluid impermeable barrier 102. As such, the fluid permeable body 120 may support and maintain the position of the fluid permeable membrane 118. The fluid permeable body 120 may include any material that may wick the fluid, such as any of the fluid permeable membrane materials disclosed herein above. For example, the fluid permeable membrane material(s) may be utilized in a more dense or rigid form than in the fluid permeable membrane 118 when used as the fluid permeable body 120. The fluid permeable body 120 may be formed from any fluid permeable material that is less deformable than the fluid permeable membrane 118. For example, the fluid permeable body 120 may include a porous polymer (e.g., nylon, polyester, polyurethane, polyethylene, polypropylene, etc.) structure or an open cell foam. In some examples, the fluid permeable body 120 may be formed from a natural material, such as cotton, wool, silk, or combinations thereof. In such examples, the material may have a coating to prevent or limit absorption of fluid into the material, such as a water repellent coating. In some examples, the fluid permeable body 120 may be formed from fabric, felt, gauze, or combinations thereof. In some examples, the fluid permeable membrane 118 may be optional. For example, the porous material 120 may include only the fluid permeable body 120. In some examples, the fluid permeable body 120 may be optionally omitted from the fluid collection device 100. For example, the porous material 120 may only include the fluid permeable membrane 118.

In a particular example, the fluid permeable membrane 118 may include first layer and a second layer. The first layer may be positioned to generally receive bodily fluid before the second layer. The first layer may include hydrophilic polypropylene or hydrophilic polyethylene (e.g., polypropylene or polyethylene including a polyethylene glycol fatty acid ester surfactant or otherwise treated to be hydrophilic) and the second layer may include bamboo. In such an example, the first layer and the second layer may have a synergistic effect that allows the first layer to quickly receive bodily fluids therein, move the bodily fluids from the first layer into the second layer, and maintain the first layer relatively dry. For instance, the hydrophilicity of the first layer allows the first layer to quickly receive bodily fluids, such as to initially receive bodily fluids that are discharged from the urethral opening of the individual. However, the bamboo second layer may exhibit a hydrophilicity that is greater than (i.e., exhibits a contact angle with water that is less than) the first layer. The greater hydrophilicity of the bamboo second layer pulls bodily fluids from the first layer and into the second layer, thereby facilitating quick transfer of bodily fluids from the first layer to the second layer. The greater hydrophilicity of the bamboo second layer also helps dry the first layer since the hydrophilic pull from the bamboo second layer removes most of the bodily fluids from the first layer. The dry first layer minimizes discomfort caused by using a fluid collection assembly including the porous material, minimizes skin degradation caused by the bodily fluids, and allows the fluid collection assembly including the porous material to be used for longer periods of time (e.g., greater than 24 hours). In this particular example, the first layer and the second layer may be positioned adjacent to a polyethylene terephthalate (“PET”) fluid permeable body since the bamboo second layer is able to transfer bodily fluids quickly and effectively into the PET fluid permeable body thereby preventing the first and second layers from becoming saturated with bodily fluids. Further, the PET fluid permeable body is able to quickly move substantially all of the bodily fluids towards a fluid outlet such that the PET fluid permeable body is substantially dry a short period of time after receiving the bodily fluids. The dry PET fluid permeable body facilitates drying of the particular first and second layers of this example. It is noted that the PET fluid permeable body may include a nonwoven material and, more particularly a vertical lapped nonwoven material, since such nonwoven materials facilitate drawing fluids into the PET fluid permeable body from the bamboo second layer and improve flow of the bodily fluids in the PET fluid permeable body towards the fluid outlet thereby facilitating drying of the porous material 118. That said, the first layer and/or the second layer may include porous materials other than hydrophilic polypropylene/polyethylene and bamboo, respectively, as discussed in more detail herein. Further, the first layer and/or the second layer may be used with an inner fluid permeable body other than PET, as discussed in more detail herein.

The fluid permeable body 120 may have a greater ability to wick fluids than the fluid permeable membrane 118, such as to move the fluid inwardly from the outer surface of the fluid collection device 100. In some examples, the wicking ability of the fluid permeable body 120 and the fluid permeable membrane 118 may be substantially the same.

In some embodiments, the fluid permeable body 120 may be a porous layer that includes a non-woven fluid permeable material, such as one or more of polyester, nylon, polypropylene, polyethylene, cellulose, and/or combinations thereof. Descriptions and aspects of non-woven material and configurations thereof are disclosed in PCT Patent Application No. PCT/US 23/24805, filed on Jun. 8, 2023, the disclosure of which is incorporated herein, in its entirety, by this reference. In some embodiments, the fluid permeable membrane 118 includes one or more of a bamboo material, a cotton material, and/or a non-woven hydrophilic material. More specifically, the fluid permeable membrane may include one or more of the bamboo material, the cotton material, a non-woven polypropylene hydrophilic material, a non-woven polyethylene hydrophilic material, a non-woven polyester hydrophilic material, and/or combinations thereof (such as a bamboo/cotton mixed material). Additional descriptions and examples of porous materials and treatments are disclosed in U.S. Provisional Patent Application No. No. 63/683,428, filed on Aug. 15, 2024 and U.S. Provisional Patent Application No. 63/568,615 filed on Mar. 22, 2024, the disclosures of each of which are incorporated herein, in their entirety, by this reference.

The fluid permeable membrane 118 and the fluid permeable body 120 may at least substantially completely fill the portions of the chamber 104 that are not occupied by the conduit 108. In some examples, the fluid permeable membrane 118 and the fluid permeable body 120 may not substantially completely fill the portions of the chamber 104 that are not occupied by the conduit 108. In such an example, the fluid collection device 100 includes a reservoir or sump 222 (FIG. 2CB) disposed in the distal receptacle 227 in the chamber 104.

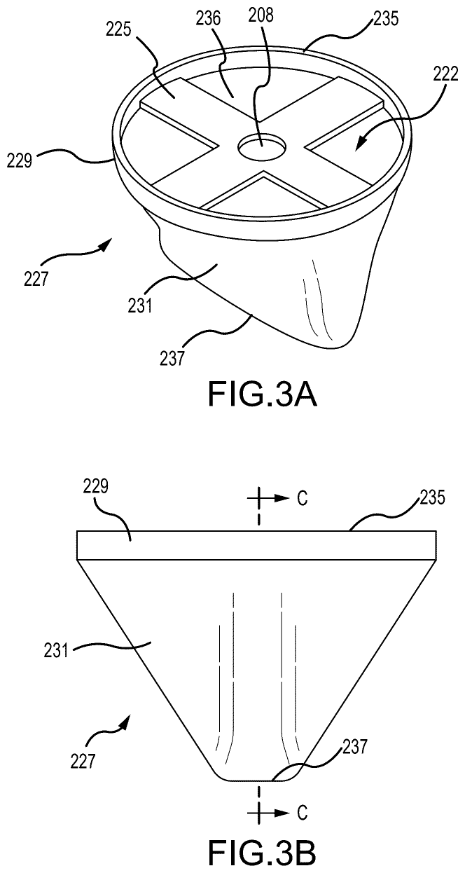

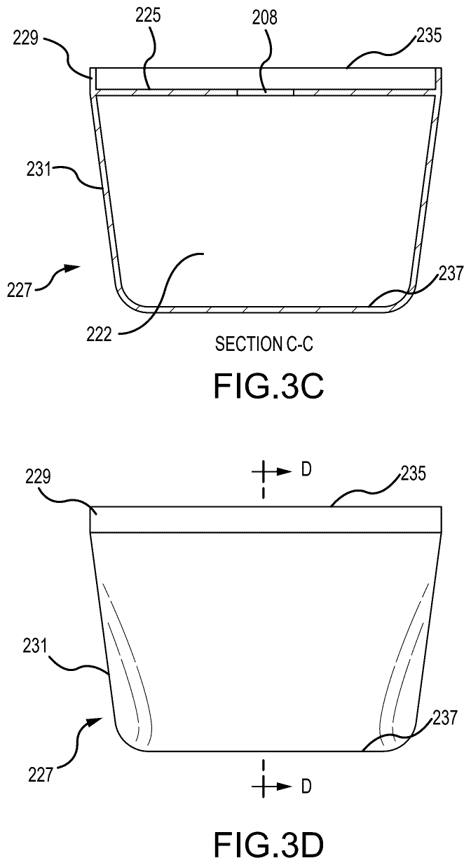

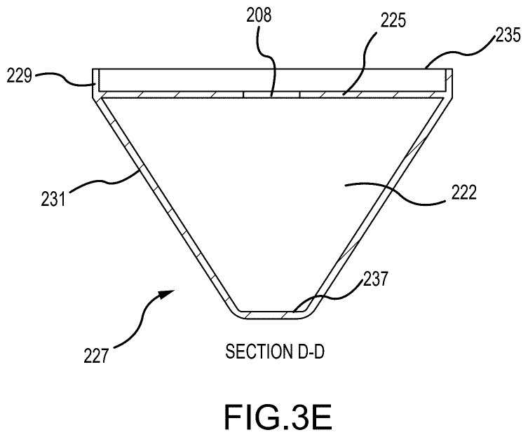

FIG. 2C is an isometric view of the conduit 108 and the distal receptacle 227 of the fluid collection device 100 removed from the fluid collection device 100 and also removed from the porous material 120. In some embodiments, the conduit 108 and the distal receptacle 227 may be assembled and secured together before being secured to the porous material 120 (and before being secured to the fluid impermeable barrier 102). FIGS. 3A-3E are views of the distal receptacle of the fluid collection device of FIG. 1A.

The distal receptacle 227 is more rigid than the fluid impermeable barrier 102. For example, in some embodiments, the distal receptacle is semi-rigid or substantially rigid. The distal receptacle 227 may be formed of a plastic material, such as polypropylene. In some embodiments, the distal receptacle 227 may be formed of one or more polypropylene, polyvinyl chloride, polystyrene, polyaramid, polyether ether ketone, polycarbonate, polyethylene (e.g., high-density polyethylene or low-density polyethylene), or combinations thereof. In some embodiments, the distal receptacle 227 is sized and dimensioned to fit within the chamber 104 in the distal end region 127 of the fluid impermeable barrier 102. For example, the distal receptacle 227 may be sized such that when the distal receptacle 227 is disposed within the chamber 104 in the distal end region 127 of the fluid impermeable barrier 102, the distal receptacle 227 is aligned with the distal end of the opening 106 or spaced distally from the distal end of the opening 106. Because the distal receptacle 227 is more rigid than the fluid impermeable barrier 102, the distal end region 127 of the fluid impermeable barrier 102 may generally conform to the shape of the outer surface of the distal receptacle 227 in many embodiments. In some embodiments (not shown), the distal receptacle 227 is sized and dimensioned such that the distal end region 127 of the fluid impermeable barrier 102 fits within the distal receptacle 227 and the distal end region 127 of the fluid impermeable barrier at least partially defines the reservoir.

The distal receptacle 227 defines a reservoir 222 in fluid communication with the opening 106 through the porous material 120. The reservoir 222 may be a region within distal receptacle 227 (and the fluid collection device 100) that is generally devoid of other materials (e.g., empty or void space). Said another way, the reservoir 222 may be a substantially unoccupied portion of the distal receptacle 227. The fluid(s) that are in the chamber 104 may flow through the fluid permeable membrane 118 and/or fluid permeable body 120 to the reservoir 222. The reservoir 222 may retain the fluid(s) therein.

The fluid(s) that are in the chamber 104 may flow through the fluid permeable membrane 118 and/or fluid permeable body 120 to the reservoir 222. The fluid impermeable barrier 102 may retain the fluid(s) in the reservoir 222. The reservoir 222 may be located in a portion of the chamber 104 that is designed to be located in a gravimetrically low point of the fluid collection device when the device is worn.

The distal receptacle 227 includes a proximal end 235 and a distal end 237. In some embodiments, the distal receptacle 227 tapers between the proximal end 235 and the distal end 237. In these and other embodiments, the distal end region 127 may generally conform to the shape of the distal receptacle 227 such that the distal end region 227 is shaped generally complementary to the distal receptacle 227 (e.g., the distal end region 127 also tapers between the opening 106 and the distal end of the fluid impermeable barrier 102). In a particular embodiment, the distal receptacle 227 is generally wedge-shaped. For example, the distal receptacle 227 may include generally opposing sidewalls 231 that angle towards one another between the proximal end 235 and the distal end 237 of the distal receptacle 227. In some embodiments, the fluid impermeable barrier 102 is substantially cylindrical, the proximal end 235 of the distal receptacle 227 is substantially circular, and the distal end 237 of the distal receptacle 225 is an elongated edge (e.g., rounded or flat elongated edge). The distal receptacle 227 may be positioned such that the elongated edge at the distal end 237 is elongated between a front region and a rear region of the fluid impermeable barrier 102 (with the front region defining the opening 106 and the rear region generally opposite to the front region).

In some embodiments, the proximal end 235 of the distal receptacle 227 includes a proximal rim 229. The proximal rim 229 may be a ring and may be generally circular. The distal receptacle 227 also may include one or more support members 225 extending at least partially across the reservoir 222 of the distal receptacle 227. The one or more support members 225 may be recessed from the proximal end 235 of the distal receptacle 227. For example, the proximal rim 229 may extend from the proximal end 237 to the one or more support members 225 and/or the support member 225 may be secured to the proximal rim 229 distal from the proximal end 235 of the distal receptacle 227. The area of the distal receptacle 227 between the proximal end 235 and the one or more support member 225 may be an assembly area sized to receive the distal end of the porous material 120 (e.g., the one or more support members 225 and the proximal rim 229 may at least partially define an assembly area). For example, the distal end region of the porous material 120 may be sized to fit within the assembly area at least partially defined by the one or more support members 225 and the proximal rim 229. In some embodiments, the distal end region of the porous material 120 is sized larger than the assembly area of the distal receptacle 227 until the distal end region is inserted into the assembly area. The proximal rim 229 of the distal receptacle 227 may compress the distal end region of the porous material 120 to secure the porous material 120 to the distal receptacle 227. In some embodiments, the distal end region of the porous material 120 may be secured to the proximal rim 229 of the distal receptacle 227 with an adhesive.

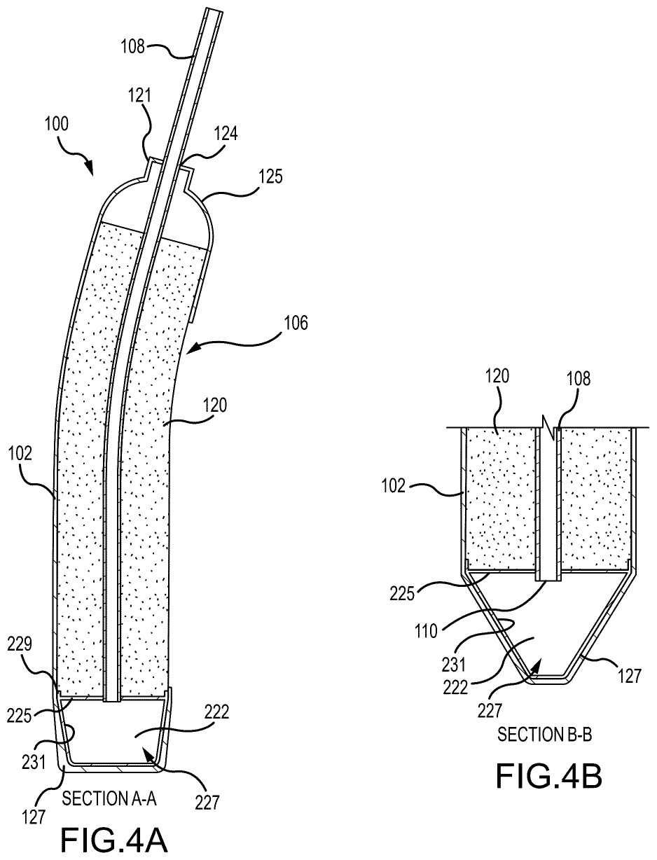

In some embodiments, the one or more support members 225 form an x shape or a +shape across the proximal end of the reservoir 222 in the distal receptacle 227. The one or more support members 225 are positioned to form openings or gaps 236 therebetween to allow fluid to flow through the porous member 120 into the reservoir 222 without being entirely obstructed by the one or more support members 225. In some embodiments, the one or more support members 225 define an additional aperture 208. The additional aperture 208 may be sized to have the conduit 108 extend therethrough. In some embodiments, the additional aperture 208 is sized to friction fit around the distal end of the conduit 108 to secure the conduit 108 to the distal receptacle 227. In some embodiments (not shown), the distal receptacle 227 may include a port protruding distally from the one or more support members 225 and defining the additional aperture 208. The port may be sized to friction fit within the inlet 110 (shown in FIG. 4B) of the conduit 108 to secure the conduit 108 to the distal receptacle 227.

The conduit 108 may be at least one of secured to or extending through the one or more support members. The conduit 108 also may be at least partially disposed in the chamber 104. The conduit 108 may be used to remove fluid from the chamber 104. The conduit 108 (e.g., a tube) includes an inlet 110 and an outlet positioned downstream from the inlet 110. The outlet may be operably coupled to a suction source, such as a vacuum pump for withdrawing fluid from the chamber through the conduit 108. For example, the conduit 108 may extend into the fluid impermeable barrier 102 from the first end region 125 and may extend to the second end region 127 to a point proximate to the reservoir 222 therein such that the inlet 110 is in fluid communication with the reservoir 122. The conduit 108 fluidly couples the chamber 104 and the reservoir 222 of the distal receptacle with the fluid storage container (not shown) or the vacuum source (not shown).

The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 108 may include silicon or latex. In some examples, the conduit 108 may include one or more portions that are resilient, such as by having one or more of a diameter or wall thickness that allows the conduit to be flexible.

Turning ahead in the drawings, FIG. 4A is a cross-sectional view of the fluid collection device 100 along A-A of FIG. 1A and FIG. 4B is a cross-sectional view of the distal end region 127 of the of the fluid collection device 100 along B-B of FIG. 1B. As shown in FIGS. 4A and 4B, the end of the conduit 108 may extend through a bore in the fluid permeable membrane 118 and/or fluid permeable body 120, such as into the reservoir 222. For example, the inlet 110 of the conduit 108 may extend into or be positioned in the reservoir 222. In the illustrated embodiment, the conduit 108 is at least partially disposed in the distal receptacle 227 and the reservoir 222 of the distal receptacle 227. The fluid collected in the fluid collection device 100 may be removed from the chamber 104 via the conduit 108.

In some examples, the inlet 110 may not extend into the reservoir 122. In such examples, the inlet 110 may be disposed within the porous material 120 (fluid permeable membrane 118 and/or fluid permeable body 120) or at a terminal end thereof. For example, an end of the conduit 108 may be coextensive with or recessed within the fluid permeable membrane 118 and/or fluid permeable body 120.

Locating the inlet 110 at or near a location expected to be the gravimetrically low point of the chamber 104 when worn by a wearer enables the conduit 108 to receive more of the fluid(s) than if the inlet 110 was located elsewhere and reduces the likelihood of pooling (e.g., pooling of the fluid(s) may cause microbe growth and foul odors). For instance, the fluid(s) in the fluid permeable membrane 118 and the fluid permeable body 120 may flow in any direction due to capillary forces. However, the fluid(s) may exhibit a preference to flow in the direction of gravity, especially when at least a portion of the fluid permeable membrane 118 and/or the fluid permeable body 120 is saturated with the fluid(s). Accordingly, one or more of the inlet 110 or the reservoir 122 may be located in the fluid collection device 100 in a position expected to be the gravimetrically low point in the fluid collection device when worn by a wearer, such as the second end region 127.

In an example, the conduit 108 is configured to be at least insertable into the chamber 104. In such an example, the conduit 108 may include one or more markers (not shown) on an exterior thereof that are located to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another example, the conduit 108 may include one or more markings thereon that are configured to facilitate correct rotation of the conduit 108 relative to the chamber 104. The one or more markings may include a line, a dot, a sticker, or any other suitable marking.

As described in more detail below, the conduit 108 is configured to be coupled to, and at least partially extend between, one or more of the fluid storage container (not shown) and the vacuum source (not shown). In an example, the conduit 108 is configured to be directly connected to the vacuum source (not shown). In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container (not shown) and the vacuum source (not shown). In some examples, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

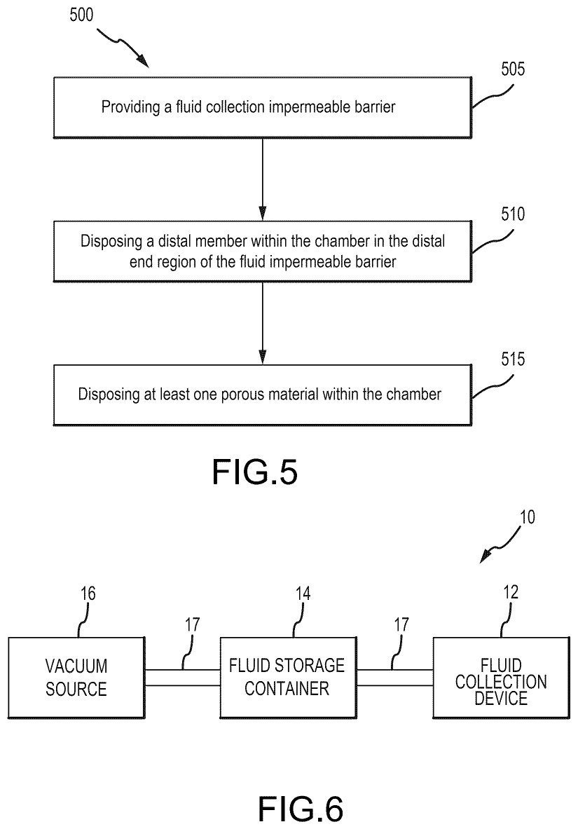

The inlet 110 and the outlet 112 are configured to fluidly couple (e.g., directly or indirectly) the vacuum source (not shown) to the chamber 104 (e.g., the reservoir 122). As the vacuum source (FIG. 6) applies a vacuum/suction in the conduit 108, the fluid(s) in the chamber 104 (e.g., at the second end region such as in the reservoir 122) may be drawn into the inlet 110 and out of the fluid collection device 100 via the conduit 108. In some examples, the conduit 108 may be frosted or opaque (e.g., black) to obscure visibility of the fluid(s) therein.

FIG. 5 is a flow diagram of a method 500 manufacturing or assembling a fluid collection device. The fluid collection device manufactured or assembled according to the method 500 may include any of the fluid collection devices disclosed herein, such as the fluid collection device 100. The method 500 includes providing 505 a fluid impermeable barrier that is generally flexible and has a proximal end region and a distal end region, the fluid impermeable barrier at least partially defining a chamber therein, an aperture in the proximal end region, and an opening extending therethrough between the distal end region and the proximal end region and configured to be positioned adjacent to a female urethra. The method 500 includes disposing 510 a distal receptacle within the chamber in the distal end region of the fluid impermeable barrier, the distal receptacle being more rigid than the fluid impermeable barrier defining a reservoir in fluid communication with the opening. The method 500 includes disposing 515 at least one porous material within the chamber such that at least a portion of the at least one porous material extends at least partially across the opening and the reservoir of the distal receptacle is in fluid communication with the opening through the at least one porous material.

In some embodiments of the method 500, the distal receptacle includes a proximal end and a distal end, and the distal receptacle tapers between the proximal end and the distal end such that the distal end region of the fluid impermeable barrier conforms to a generally tapered shape when the distal receptacle is disposed within the chamber in the distal end region of the fluid impermeable barrier. In these and other embodiments of the method 500, the distal receptacle is generally wedge-shaped such that the distal end region of the fluid impermeable barrier conforms to a generally wedge shape complementary to the distal receptacle when the distal receptacle is disposed within the chamber in the distal end region of the fluid impermeable barrier. In these and other embodiments of the method 500, the fluid impermeable barrier is substantially cylindrical, the proximal end of the distal receptacle is substantially circular, and the distal end of the distal receptacle is an elongated edge such that the distal end region of the fluid impermeable barrier conforms to a generally wedge shape with an elongated edge complementary to the distal receptacle when the distal receptacle is disposed within the chamber in the distal end region of the fluid impermeable barrier. In some embodiments of the method 500, the fluid impermeable barrier includes a front region defining the opening and a rear region generally opposite to the front region, and the distal receptacle is disposed in the distal end region such that the elongated edge is elongated between the front region and the rear region of the fluid impermeable barrier.

The method 500 also may include acts of extending a conduit through a proximal neck of the fluid impermeable barrier at least partially defining the aperture and extending proximally from the proximal end region of the fluid impermeable barrier, extending conduit extending through the at least one porous material, disposing an inlet of the conduit at least proximate to the reservoir such that the conduit is in fluid communication with the reservoir, and adhesively bonding the conduit to the proximal neck. In these and other embodiments, disposing an inlet of the conduit at least proximate to the reservoir such that the conduit is in fluid communication with the reservoir may include securing the conduit to one or more support members of the distal receptacle extending at least partially across the reservoir of the distal receptacle. More particularly, securing the conduit to one or more support members of the distal receptacle extending at least partially across the reservoir of the distal receptacle may include extending the conduit through an additional aperture defined by the one or more support members of the distal receptacle. In some embodiments, the method 500 may include first securing the conduit to the distal receptacle and then extending the conduit through the porous material. Alternatively, the method 500 may include first extending the conduit through the porous material and then securing the conduit to the distal receptacle. In either of these embodiments, once secured to one another, the subassembly of the distal receptacle, the conduit, and the porous material may be inserted into the chamber of the fluid impermeable barrier with the distal receptacle in the distal end region and/or the conduit extending through the aperture at the proximal end region.

In many embodiments of the method 500, the at least one porous material includes a vertical non-woven foam and wicking material covering the vertical non-woven foam across at least the opening of the fluid impermeable barrier, the wicking material including a bamboo or hydrophilic polypropylene material.

Acts of the method 500 are for illustrative purposes. For example, unless otherwise noted, the acts of the method 500 may be performed in different orders, split into multiple acts, modified, supplemented, or combined.

FIG. 6 is a block diagram of a system 10 for fluid collection, according to an embodiment. The system 10 includes a fluid collection device 12, a fluid storage container 14, and a vacuum source 16 (e.g., portable vacuum device). The fluid collection device 12, the fluid storage container 14, and the vacuum source 16 may be fluidly coupled to each other via one or more conduits 17. For example, fluid collection device 12 may be operably coupled to one or more of the fluid storage container 14 or the vacuum source 16 via the conduit 17. Fluid (e.g., urine or other bodily fluids) collected in the fluid collection device 12 may be removed from the fluid collection device 12 via the conduit 17 which protrudes into the fluid collection device 12. For example, an inlet of the conduit 17 may extend into the fluid collection device 12, such as to a reservoir therein. The outlet of the conduit 17 may extend into the fluid collection device 12 or the vacuum source 16. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the vacuum source 16 either directly or indirectly. The suction force may be applied indirectly via the fluid storage container 14. For example, the outlet of the conduit 17 may be disposed within the fluid storage container 14 and an additional conduit 17 may extend from the fluid storage container 14 to the vacuum source 16. Accordingly, the vacuum source 16 may apply suction to the fluid collection device 12 via the fluid storage container 14. The suction force may be applied directly via the vacuum source 16. For example, the outlet of the conduit 17 may be disposed within the vacuum source 16. An additional conduit 17 may extend from the vacuum source 16 to a point outside of the fluid collection device 12, such as to the fluid storage container 14. In such examples, the vacuum source 16 may be disposed between the fluid collection device 12 and the fluid storage container 14.

The fluid collection device 12 may be similar or identical to any of the fluid collection devices disclosed herein (e.g., 100) in one or more aspects. The fluid collection device 12 may be shaped and sized to be positioned adjacent to a female urethra. For example, the fluid collection device 12 may include a fluid impermeable barrier at least partially defining a chamber (e.g., interior region) of the fluid collection device 12. The fluid impermeable barrier also defines an opening extending therethrough from the external environment. The opening may be positioned adjacent to a female urethra. The fluid collection device 12 may include a fluid permeable membrane disposed within the fluid impermeable barrier. The fluid collection device 12 may include porous material disposed in the chamber such as one or more of a fluid permeable body and a fluid permeable membrane. The fluid collection device 12 may include the distal receptacle disposed in the distal end region. The conduit 17 may extend into the fluid collection device 12 at a first end (e.g., proximal) region, through one or more of the fluid impermeable barrier, fluid permeable membrane, or the fluid permeable body to a second end (e.g., distal) region of the fluid collection device 12. The conduit 17 includes an inlet and an outlet, the outlet being fluidly coupled to the fluid storage container and the inlet being positioned in a portion of the chamber selected to be at a gravimetrically low point of the fluid collection device when worn.

The fluid storage container 14 is sized and shaped to retain a fluid therein. The fluid storage container 14 may include a bag (e.g., drainage bag), a bottle or cup (e.g., collection jar), or any other enclosed container for storing bodily fluid(s) such as urine. In some examples, the conduit 17 may extend from the fluid collection device 12 and attach to the fluid storage container 14 at a first point therein. An additional conduit 17 may attach to the fluid storage container 14 at a second point thereon and may extend and attach to the vacuum source 16. Accordingly, a vacuum (e.g., suction) may be drawn through fluid collection device 12 via the fluid storage container 14. Fluid, such as urine, may be drained from the fluid collection device 12 using the vacuum source 16.

The vacuum source 16 may include one or more of a manual vacuum pump, electric vacuum pump, a diaphragm pump, a centrifugal pump, a displacement pump, a magnetically driven pump, a peristaltic pump, or any pump configured to produce a vacuum. The vacuum source 16 may provide a vacuum or suction to remove fluid from the fluid collection device 12. In some examples, the vacuum source 16 may be powered by one or more of a power cord (e.g., connected to a power socket), one or more batteries, or even manual power (e.g., a hand operated vacuum pump). In some examples, the vacuum source 16 may be sized and shaped to fit outside of, on, or within the fluid collection device 12. For example, the vacuum source 16 may include one or more miniaturized pumps or one or more micro pumps. The vacuum sources 209 disclosed herein may include one or more of a switch, a button, a plug, a remote, or any other device suitable to activate the vacuum source 16.

As used herein, the terms “about” or “substantially” refer to an allowable variance of the term modified by “about” by ±10% or ±5%. Further, the terms “less than,” “or less,” “greater than,” “more than,” or “or more” include as an endpoint, the value that is modified by the terms “less than,” “or less,” “greater than,” “more than,” or “or more.” While various aspects and embodiments have been disclosed herein, other aspects and embodiments are contemplated. The various aspects and embodiments disclosed herein are for purposes of illustration and are not intended to be limiting.

1. A fluid collection device, comprising:
a fluid impermeable barrier that is generally flexible and has a proximal end region and a distal end region, the fluid impermeable barrier at least partially defining a chamber therein, an aperture in the proximal end region, and an opening extending therethrough between the distal end region and the proximal end region and configured to be positioned adjacent to a female urethra;
at least one porous material disposed in the chamber, at least a portion of the at least one porous material positioned to extend across at least partially across the opening; and
a distal receptacle that is more rigid than the fluid impermeable barrier and positioned within the chamber in the distal end region of the fluid impermeable barrier or has the distal end region of the fluid impermeable barrier positioned within the distal receptacle.

2. The fluid collection device of claim 1, wherein the distal receptacle is positioned within the chamber in the distal end region of the fluid impermeable barrier and the distal receptacle defines a reservoir in fluid communication with the opening through the at least one porous material.

3. The fluid collection device of claim 2, wherein the distal receptacle includes a proximal end and a distal end, the distal receptacle tapering between the proximal end and the distal end and the distal end region is shaped generally complementary to the distal receptacle.

4. The fluid collection device of claim 3, wherein the distal receptacle is generally wedge-shaped.

5. The fluid collection device of claim 3 wherein the fluid impermeable barrier is substantially cylindrical, the proximal end of the distal receptacle is substantially circular, and the distal end of the distal receptacle is an elongated edge.

6. The fluid collection device of claim 5, wherein the fluid impermeable barrier includes a front region defining the opening and a rear region generally opposite to the front region, and wherein the distal receptacle is positioned such that the elongated edge is elongated between the front region and the rear region of the fluid impermeable barrier.

7. The fluid collection device of claim 2, further comprising:
wherein the fluid impermeable barrier includes a proximal neck extending from the proximal end region and at least partially defining the aperture; and
a conduit extending through the proximal neck, the aperture, and the at least one porous material, the conduit being bonded to the proximal neck with an adhesive and having an inlet disposed at least proximate to the reservoir such that the conduit is in fluid communication with the reservoir.

8. The fluid collection device of claim 7, wherein the distal receptacle includes one or more support members extending at least partially across the reservoir of the distal receptacle, the conduit being at least one of secured to or extending through the one or more support members.

9. The fluid collection device of claim 8, wherein the one or more support members of the distal receptacle define an additional aperture and the conduit extends through the additional aperture.

10. The fluid collection device of claim 1, wherein the at least one porous material includes a vertical non-woven foam and wicking material covering the vertical non-woven foam across at least the opening of the fluid impermeable barrier, the wicking material including a bamboo or hydrophilic polypropylene material.

11. The fluid collection device of claim 1, wherein the distal receptacle is substantially rigid and the distal end region of the fluid impermeable barrier conforms to a shape of the distal receptacle.

12. A fluid collection system, comprising:
a fluid storage container configured to hold a fluid;
a fluid collection device comprising:
a fluid impermeable barrier that is generally flexible and has a proximal end region and a distal end region, the fluid impermeable barrier at least partially defining a chamber therein, an aperture in the proximal end region, and an opening extending therethrough between the distal end region and the proximal end region and configured to be positioned adjacent to a female urethra;
at least one porous material disposed in the chamber, at least a portion of the at least one porous material positioned to extend across at least partially across the opening; and
a distal receptacle that is more rigid than the fluid impermeable barrier and positioned within the chamber in the distal end region of the fluid impermeable barrier or has the distal end region of the fluid impermeable barrier positioned within the distal receptacle; and
a vacuum source fluidly coupled to one or more of the fluid storage container or the fluid collection device via the conduit, the vacuum source configured to draw fluid from the fluid collection device via the conduit.

13. A method of assembling a fluid collection device, the method comprising:
providing a fluid impermeable barrier that is generally flexible and has a proximal end region and a distal end region, the fluid impermeable barrier at least partially defining a chamber therein, an aperture in the proximal end region, and an opening extending therethrough between the distal end region and the proximal end region and configured to be positioned adjacent to a female urethra;
disposing a distal receptacle within the chamber in the distal end region of the fluid impermeable barrier, the distal receptacle being more rigid than the fluid impermeable barrier defining a reservoir in fluid communication with the opening; and
disposing at least one porous material within the chamber such that at least a portion of the at least one porous material extends at least partially across the opening and the reservoir of the distal receptacle is in fluid communication with the opening through the at least one porous material.

14. The method of claim 13, wherein the distal receptacle includes a proximal end and a distal end, the distal receptacle tapering between the proximal end and the distal end such that the distal end region of the fluid impermeable barrier conforms to a generally tapered shape when the distal receptacle is disposed within the chamber in the distal end region of the fluid impermeable barrier.

15. The method of claim 14, wherein the distal receptacle is generally wedge-shaped such that the distal end region of the fluid impermeable barrier conforms to a generally wedge shape complementary to the distal receptacle when the distal receptacle is disposed within the chamber in the distal end region of the fluid impermeable barrier.

16. The method of claim 14 wherein the fluid impermeable barrier is substantially cylindrical, the proximal end of the distal receptacle is substantially circular, and the distal end of the distal receptacle is an elongated edge such that the distal end region of the fluid impermeable barrier conforms to a generally wedge shape with an elongated edge complementary to the distal receptacle when the distal receptacle is disposed within the chamber in the distal end region of the fluid impermeable barrier.

17. The method of claim 16, wherein the fluid impermeable barrier includes a front region defining the opening and a rear region generally opposite to the front region, and wherein the distal receptacle is disposed in the distal end region such that the elongated edge is elongated between the front region and the rear region of the fluid impermeable barrier.

18. The method of claim 13, further comprising:
extending a conduit through a proximal neck of the fluid impermeable barrier at least partially defining the aperture and extending proximally from the proximal end region of the fluid impermeable barrier;
extending conduit extending through the at least one porous material;
disposing an inlet of the conduit at least proximate to the reservoir such that the conduit is in fluid communication with the reservoir; and
adhesively bonding the conduit to the proximal neck.

19. The method of claim 18, wherein disposing an inlet of the conduit at least proximate to the reservoir such that the conduit is in fluid communication with the reservoir includes securing the conduit to one or more support members of the distal receptacle extending at least partially across the reservoir of the distal receptacle.

20. The method of claim 19, wherein securing the conduit to one or more support members of the distal receptacle extending at least partially across the reservoir of the distal receptacle includes extending the conduit through an additional aperture defined by the one or more support members of the distal receptacle.

21. The method of claim 13, wherein the at least one porous material includes a vertical non-woven foam and wicking material covering the vertical non-woven foam across at least the opening of the fluid impermeable barrier, the wicking material including a bamboo or hydrophilic polypropylene material.
